(19) United States
(12) Patent Application Publication
(10) Pub. No.: US 2025/0041294 A1
(43) Pub. Date: Feb. 6, 2025

(54) Synergistic compositions for use in the treatment of cancer

(71) Applicant: Sartar Therapeutics Oy, Helsinki (FI)

(72) Inventors: Harri Sihto, Helsinki (FI); Kirsi Toivanen, Helsinki (FI); Tom Böhling, Helsinki (FI)

(21) Appl. No.: 18/716,965

(22) PCT Filed: Dec. 7, 2022
(86) PCT No.: PCT/FI2022/050813

§ 371 Date: Jun. 6, 2024

(30) Foreign Application Priority Data
Dec. 7, 2021 (FI) 20216252

(51) Int. Cl. A61K 31/501; A61K 31/404; A61K 31/428; A61K 31/436; A61K 31/4985; A61K 31/506; A61K 31/519; A61K 31/635

(52) U.S. Cl. CPC A61K 31/501; A61K 31/404; A61K 31/428; A61K 31/436; A61K 31/4985; A61K 31/506; A61K 31/519; A61K 31/635

(57) ABSTRACT

The present invention is related to a composition comprising a phosphodiesterase 3A modulator compound in synergistic combination with an inhibitor compound selected from the group consisting of: an inhibitor of Bcl-2 family proteins, a mTOR inhibitor, a histone deacetylase (HDAC) inhibitor, a DNA-dependent protein kinase (DNA-PK) inhibitor, an inhibitor of integrin alpha 2 protein, a NEDD8-activating enzyme (NAE) inhibitor and a PAK4 inhibitor for use in the treatment of cancer.

### FIELD

The field of the present disclosure relates to cancer biology. The present disclosure further relates to multi-drug combinations for use in the treatment of cancer.

### BACKGROUND

Nearly 20 million new cancer cases were diagnosed, and 10 million patients died of cancer world widely in 2020. A global cancer incidence is estimated to increase up to 28.4 million cases in 2040. Although, cancer therapies have improved during past decades, many cancers do not respond or they become resistant to current therapies. One approach to meet this challenge is to develop personalized medicine approaches to target cancer driving mechanisms, which are important for the survival, proliferation or dissemination of cancer cells. For example, targeting on overexpressed HER2 receptor with HER2-specific antibodies or antibody-drug conjugates has significantly improved outcome in metastatic breast cancer. Similarly, small molecule inhibitors such as the inhibitors targeting mutated EGFR tyrosine kinase receptor in lung cancer or ABR-ABL1 fusion-gene in chronic myeloid leukemia are efficacious cancer therapies.

Phosphodiesterase 3 (PDE3) proteins are promising new therapy targets in cancer (WO2015055898, Pulkka O, et al. 2019). PDE3s are enzymes, which catalyze hydrolysis of second messengers cAMP and cGMP to AMP and GMP in cells. In healthy tissues, PDE3A isoforms are commonly expressed in cardiac myocytes, vascular smooth muscle, platelets, oocytes and placenta. PDE3B expression is common in hepatocytes, adipocytes and vascular smooth muscle. PDE3 enzyme inhibitors such as Anagrelide, Milrinone and Cilostazol are used to treat essential thrombocytosis, heart failure and intermittent claudication in peripheral vascular disease.

Expression of PDE3A is detected frequently in gastrointestinal stromal tumor (GIST) and in other cancer types. Treatment of PDE3s expressing cancer cells with target specific compounds such as Anagrelide, nauclefine, 17-beta-estradiol related hormones, (6S)-5-[4′-Fluoro-2-(trifluoromethyl) biphenyl-4-yl]-6-methyl-3,6-dihydro-2H-1,3,4-oxadiazin-2-one, also known assBAY2666605, and 6-(4-(diethylamino)-3-nitrophenyl)-5-methyl-4,5-dihydropyridazin-3 (2H)-one, also known as DNMDP, kills and reduces growth and proliferation of tumor cells in vitro and in vivo (WO2015055898, Pulkka O, et al. 2019, Nazir M, et al. 2017, de Waal L, et al. 2016). Binding of these molecules to PDE3A induces interaction between PDE3A and Schlafen 12 (SLFN12) protein, leading to suppression of cell growth or to cell death. The interaction of SLFN12 with PDE3A stabilizes SLFN12, which has normally fast turn-over within cells, and leads to elevated binding of SLFN12 to ribosomes and blocking of protein translation of anti-apoptotic proteins BCL2 and MCL1. Further, expression of other cell cycle or apoptosis regulating genes, such as TNF-alfa, DR4, DR5, Bcl-2, Bcl-Xl, Cyclin D1 and p21, are altered after Anagrelide treatment. In addition, the SLFN12 interaction with PDE3A increases RNase activity of SLFN12 that is required for DNMDP response in cells. Expression of PDE3B in cancer cells complements PDE3A as a drug target (Pulkka O, et al. 2019, Wu X, et al. 2020).

Gene and protein expression level of PDE3A in cancer cells correlates positively with an anticancer efficacy of PDE3A modulating agents (Wu X, et al. 2020). EP3411037 discloses the use of PDE3A modulators in the treatment of cancer and in cancer diagnostics. However, a low PDE3A expression reduces efficacy of PDE3A specific anticancer therapy and therefore novel strategies are still needed.

PDE3A expressing cancer cell lines can be sensitized to Anagrelide by interferon treatment. IFN-α and IFN-γ induce SLFN12 expression in cells and therefore they enable Anagrelide induced cell death through the PDE3A-SLFN12 complex in PDE3A expressing cells. In addition, Anagrelide treatment increases expression of cell death signaling pathway genes TNF-α, and death receptors DR4 and DR5 in the cells. Treatment of Bel7404 cells with DR4 and DR5 ligands TNF-α and TRAIL shows synergy with Anagrelide (An R, et al. 2019). Combination therapies might thus present an avenue for improved cancer therapies.

### SUMMARY

The invention is defined by the features of the independent claims. Some specific embodiments are defined in the dependent claims.

As described below, the present invention discloses synergistic combinations of phosphodiesterase 3A modulators and specific protein inhibitors for use in the treatment of cancer.

Accordingly, the present invention provides a phosphodiesterase 3A modulator compound or a pharmaceutically acceptable salt thereof in synergistic combination with an inhibitor compound selected from the group consisting of: an inhibitor of Bcl-2 family proteins, a mTOR inhibitor, a histone deacetylase (HDAC) inhibitor, a DNA-dependent protein kinase (DNA-PK) inhibitor, an inhibitor of integrin alpha 2 protein, a NEDD8-activating enzyme (NAE) inhibitor, a PAK4 inhibitor and a pharmaceutically acceptable salt thereof; for use in the treatment of cancer.

### BRIEF DESCRIPTION OF THE DRAWINGS

FIG. 1 shows characterization of cell lines. A) Expression of PDE3A, SLFN12 and actin in cell lines was analyzed by western blot. B) Immunohistochemical staining of PDE3A in cell lines confirmed expression of PDE3A in GIST882, SA4 and GOT3. C) IC₅₀ values for Anagrelide were defined in the three Anagrelide sensitive cell lines.

FIG. 2. Cilostazol, a PDE3-specific enzyme inhibitor, does not show synergy with Bcl-family inhibitor A1155463.

FIG. 3. Efficacy of Anagrelide alone and in combination with Bcl-family inhibitor A1155463 in A) GIST882, B) SA4 and C) GOT3 cell lines. DMSO treated cells assay represent negative control group of treatment.

FIG. 4. Efficacy of Anagrelide alone and in combination with Bcl-family inhibitor Navitoclax in A) GIST882, B) SA4, and C) GOT3 cell lines. DMSO treated cells assay represent negative control group of treatment.

FIG. 5. Efficacy of 3-hydroxy anagrelide alone and in combination with Bcl-family inhibitor A1155463 in A) GIST882, B) SA4 and C) GOT3 cell lines. DMSO treated cells assay represent negative control group of treatment.

FIG. 6. Efficacy of 3-hydroxy anagrelide alone and in combination with Bcl-family inhibitor Navitoclax in A) GIST882, B) SA4 and C) GOT3 cell lines. DMSO treated cells assay represent negative control group of treatment.

FIG. 7. Efficacy of DMNDP alone and in combination with Bcl-family inhibitor A1155463 in A) GIST882, B) SA4 and C) GOT3 cell lines. DMSO treated cells assay represent negative control group of treatment.

FIG. 8. Efficacy of DNMDP alone and in combination with Bcl-family inhibitor Navitoclax in A) GIST882, B) SA4 and C) GOT3 cell lines. DMSO treated cells assay represent negative control group of treatment.

### DETAILED DESCRIPTION OF THE INVENTION

As used herein, the terms “subject,” “individual,” “host,” and “patient,” are used interchangeably herein to refer to an animal being treated with one or more exemplary compounds as taught herein, including, but not limited to, simians, humans, avians, felines, canines, equines, rodents, bovines, porcines, ovines, caprines, mammalian farm animals, mammalian sport animals, and mammalian pets. A suitable subject for various embodiments can be any animal, including a human, that is suspected of having, has been diagnosed as having, or is at risk of developing a disease that can be ameliorated, treated or prevented by administration of one or more exemplary compounds as described herein

As used herein, the term “treatment” or “treating” refers to administration of the compound of the invention to a subject, e.g., a mammal or human subject, for purposes which include not only complete cure, but also prophylaxis, amelioration, or alleviation of a disorder or symptoms related to a pathological condition. The therapeutic effect may be assessed by monitoring the symptoms of a patient, biomarkers in blood, a size of lesion or solid tumor, number of metastases and/or or the length of survival of the patient.

As used herein, the terms “administering” or “administration” to a subject of a therapeutic agent, composition or compound as described herein includes any route of introducing or delivering to the subject the composition or compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

By “agent” is meant any small molecule chemical compound, antibody, nucleic acid, or polypeptide, or fragments thereof.

By “phosphodiesterase 3A” and by “PDE3A” is meant a protein or fragment thereof as defined in WO2015055898.

By “Schlafen 12” and by “SLFN12” is meant a protein or fragment thereof as defined in EP3411037.

As used herein, the term “modulator” refers to any agent that binds to a polypeptide and alters a biological function or activity of the polypeptide. A modulator includes, without limitations, agents that reduce or eliminate a biological function or activity of a polypeptide. A modulator further includes, without limitations, agents that increase or decrease binding of a polypeptide to another agent. For example a modulator can promote binding of a polypeptide to another polypeptide.

The present invention is based on the finding that phosphodiesterase 3A modulators have synergistic effects on cancer cells with certain protein inhibitors. The disclosure provides novel combinations of these active substances.

Accordingly, the present invention provides a composition comprising a phosphodiesterase 3A modulator compound in synergistic combination with an inhibitor compound for use in the treatment of cancer. The inhibitor is selected from the group consisting of: inhibitors of Bcl-2 family proteins, mammalian target of rapamycin (mTOR) inhibitors, histone deacetylase (HDAC) inhibitors, DNA-dependent protein kinase (DNA-PK) inhibitors, inhibitors of integrin alpha 2 protein, NEDD8-activating enzyme (NAE) inhibitors and p21-activated kinase 4 (PAK4) inhibitors.

In some embodiments, said phosphodiesterase 3A modulator compound is selected from the group consisting of: anagrelide (CAS No: 68475-42-3), 3-hydroxy anagrelide (CAS No: 733043-41-9), nauclefine (CAS No: 57103-51-2), BRD9500 (Cas No: 1630760-75-6), DNMDP (CAS No: 328104-79-6), (R)-DNMDP, BAY2666605 (CAS No: 2275774-60-0), and pharmaceutically acceptable salts thereof. Other phosphodiesterase 3A modulators are known from EP3411037, particularly Compounds 1-6 of EP3411037. Preferably, said phosphodiesterase 3A modulator compound is anagrelide, 3-hydroxy anagrelide, BRD9500, BAY2666605, or a pharmaceutically acceptable salt thereof. Without wishing to be bound by a theory, the preferred phosphodiesterase 3A modulators of the invention induce formation of PDE3A-Schlafen 12 interaction in cancer cells (Garvie G W, et al. 2021; Lewis T A, et al., 2019).

In some embodiments, the synergistic combination comprises an inhibitor of Bcl-2 family proteins. Bcl-2 family proteins include proteins that either promote or inhibit apoptosis and control apoptosis by governing mitochondrial outer membrane permeabilization. Preferably, said Bcl-2 family protein is selected from the group consisting of: A-1155463 (CAS No: 1235034-55-5), ABT-263 (navitoclax, CAS No: 923564-51-6), A-1331852 (CAS No: 1430844-80-6), AT-101 (CAS No: 90141-22-3), WEHI-539 (CAS No: 1431866-33-9), gambogic acid (CAS No: 2752-65-0), A-1210477 (CAS No: 1668553-26-1), (S)-gossypol acetic acid (CAS No: 1189561-66-7), apogossypol (CAS No: 475-56-9) and ABT-737 (CAS 852808-4-9). Accordingly in some synergistic combination, the said phosphodiesterase 3A modulator compound is anagrelide, 3-hydroxy anagrelide, or BRD9500, BAY2666605, or a pharmaceutically acceptable salt thereof and the inhibitor of Bcl-2 family proteins is A-1155463, ABT-263 (navitoclax), or a pharmaceutically acceptable salt thereof.

In other embodiments, the synergistic combination includes a mammalian target of rapamycin (mTOR) inhibitor. mTOR is also referred to as mechanistic target of rapamycin or FK506-binding protein 12-rapamycin-associated protein 1. mTOR functions as a serine/threonine protein kinase that regulates cell growth, cell proliferation, cell motility, cell survival, protein synthesis, autophagy, and transcription. Preferably, mTOR inhibitor is selected from the group consisting of rapamycin (CAS No: 53123-88-9), sirolimus, everolimus (CAS No: 159351-69-6), ridaforolimus (CAS No: 572924-54-0), temsirolimus (CAS No: 162635-4-3), CC-115 (CAS No: 1228013-15-7), Torin 1 (CAS No: 1222998-36-8) and Torin 2 (CAS No: 1223001-51-1). Accordingly in some synergistic combinations, said phosphodiesterase 3A modulator compound is anagrelide, 3-hydroxy anagrelide, BRD9500, BAY2666605, or a pharmaceutically acceptable salt thereof and the mTOR inhibitor is ridaforolimus, temsirolimus, sirolimus, everolimus, or a pharmaceutically acceptable salt thereof.

In other embodiments, a Histone deacetylase (HDAC) inhibitor is included in the synergistic combination. HDACs are a class of enzymes that remove acetyl groups from ε-N-acetyl lysine amino acid on a histone. Preferably, HDAC inhibitor is selected from the group consisting of: vorinostat (CAS No: 149647-78-9), entinostat (CAS No: 209783-80-2), panobinostat (CAS No: 404950-80-7), mocetinostat (CAS No: 726169-73-9), belinostat (CAS No: 414864-00-9), ricolinostat (CAS No: 1316214-52-4), romidepsin (CAS No: 128517-7-7), givinostat (CAS No: 497833-27-9), dacinostat (CAS No: 404951-53-7), quisinostat (CAS No: 875320-29-9), pracinostat (CAS No: 929016-96-6), resminostat (CAS No: 864814-88-0), droxinostat (CAS No: 99873-43-5), abexinostat (CAS No: 783355-60-2), RGFP966 (CAS No: 1396841-57-8), AR-42 (CAS No: 935881-37-1), PCI34051 (CAS No: 950762-95-5), trichostatin A (CAS No: 58880-19-6), SB939 (CAS No: 929016-96-6), CI994 (CAS No: 112522-64-2), CUDC-907 (CAS No: 1339928-25-4), tubacin (CAS No: 537049-40-4), chidamide (CAS No: 743420-2-2), RG2833 (CAS No: 1215493-56-3), M344 (CAS No: 251456-60-7), MC1568 (CAS No: 852475-26-4), tubastatin A (CAS No: 1252003-15-8), scriptaid (CAS No: 287383-59-9), valproic acid (CAS No: 99-66-1), sodium phenylbutyrate (CAS No: 1716-12-7), tasquinimod (254964-60-8), kevetrin (CAS No: 500863-50-3), HPOB (CAS No: 1429651-50-2), 4SC-202 (CAS No: 1186222-89-8), TMP269 (CAS No: 1314890-29-3), CAY10603 (CAS No: 1045792-66-2), BRD73954 (CAS No: 1440209-96-0), BG45 (CAS No: 926259-99-6), LMK-235 (CAS No: 1418033-25-6), nexturastat A (CAS No: 1403783-31-2), CG200745 (CAS No: 936221-33-9), CHR2845 (CAS No: 914382-60-8) and CHR3996 (CAS No: 1256448-47-1). Accordingly in some synergistic combinations, said phosphodiesterase 3A modulator compound is anagrelide, 3-hydroxy anagrelide, BRD9500, BAY2666605, or a pharmaceutically acceptable salt thereof and the HDAC inhibitor is quisinostat or a pharmaceutically acceptable salt thereof.

In some embodiments, a DNA-dependent protein kinase (DNA-PK) inhibitor is included in the synergistic combination. Preferably, DNA-PK inhibitor is selected from the group consisting of: NU7441 (CAS No: 503468-95-9), NU7026 (CAS No: 154447-35-5), KU-0060648 (CAS No: 881375-00-4), LTURM34 (CAS No: 1879887-96-3), CC-115 (CAS No: 1228013-15-7), PIK-90 (CAS No: 677338-12-4), Wortmannin (CAS No: 19545-26-7), LY3023414 (CAS No: 1386874-6-1), M3814 (CAS No: 1637542-33-6), SF2523 (CAS No: 1174428-47-7) and compound 401 (CAS No: 168425-64-7). Accordingly in some synergistic combinations, said phosphodiesterase 3A modulator compound is anagrelide, 3-hydroxy anagrelide, BRD9500, BAY2666605, or a pharmaceutically acceptable salt thereof and the DNA-PK inhibitor is CC-115 or a pharmaceutically acceptable salt thereof.

Some synergistic combinations include an integrin alpha 2 protein inhibitor. Preferably, the integrin alpha 2 protein inhibitor is selected, without limitations, from the group consisting of: E7820 (CAS No: 289483-69-8), BTT-3033 (CAS No.: 1259028-99-3), BTT-3014, BTT-3016 and Vatelizumab (CAS No. 1238217-55-4). Accordingly in some embodiments, said phosphodiesterase 3A modulator compound is anagrelide, 3-hydroxy anagrelide, BRD9500, BAY2666605, or a pharmaceutically acceptable salt thereof and the inhibitor of integrin alpha 2 protein is E7820 or a pharmaceutically acceptable salt thereof. Other synergistic combinations include a NEDD8-activating enzyme (NAE) inhibitor. Preferably, said NAE inhibitor is pevonedistat (MLN4924, CAS No: 905579-51-3). Accordingly in some embodiments, said phosphodiesterase 3A modulator compound is anagrelide, 3-hydroxy anagrelide, BRD9500, BAY2666605, or a pharmaceutically acceptable salt thereof and the NAE inhibitor is pevonedistat (MLN4924) or a pharmaceutically acceptable salt thereof.

In some embodiments, the synergistic combination includes a PAK4 inhibitor. Preferably, the PAK4 inhibitor is selected from the group consisting of: KPT-9274 (CAS No: 1643913-93-2), PF-03758309 (CAS No: 898044-15-0), IPA-3 (CAS No: 42521-82-4), FRAXIQ36, LCH-7749944 (CAS No: 796888-12-5), glaucambinone, KY-04031 (CAS No: 468056-29-3), KY-04045 (CAS No: 1223284-75-0), Inkal (Genbank ID: 389119), GL-1196 (CAS No: 591242-70-5) and GNE-2861 (CAS No: 1394121-5-1). Accordingly in some synergistic combinations, said phosphodiesterase 3A modulator compound is anagrelide, 3-hydroxy anagrelide, BRD9500, BAY2666605, or a pharmaceutically acceptable salt thereof and the PAK4 inhibitor is PF-03758309 or a pharmaceutically acceptable salt thereof.

The present invention further relates to administering the said synergistic combination to a subject. According to some embodiments of the invention, a first dose of the phosphodiesterase 3A modulator compound and a first dose of the inhibitor compound are simultaneously administered to the subject. In other embodiments, a first dose of the phosphodiesterase 3A modulator compound and a first dose of the inhibitor compound are administered sequentially, preferably in 24 hours. In some embodiments, the phosphodiesterase 3A modulator compound and the inhibitor compound are formulated in one pharmaceutical composition. In other embodiments, the phosphodiesterase 3A modulator and the inhibitor compound are formulated in separate pharmaceutical compositions.

Said composition may contain a pharmaceutically acceptable buffer, carrier, preservative or adjuvant. The phrase “pharmaceutically acceptable” refers herein to compositions that are physiologically tolerable and do not typically produce an allergic or similar reaction, when administered to a patient. Such compositions can be prepared for storage by mixing the active agent(s) having the desired degree of purity with optional physiologically acceptable carriers, preservatives, excipients, or stabilizers (Remington's Pharmaceutical Sciences, 22nd edition, Allen, Loyd V., Jr, Ed., (2012)), in any dosage form suitable. Said pharmaceutical composition may also be formulated for sustained-release, delayed-release, or timed-release, or said pharmaceutical composition is a blend of sustained-release and immediate-release formulations.

In some embodiments, said phosphodiesterase 3A modulator compound is anagrelide or a pharmaceutically acceptable salt thereof. Preferably, said pharmaceutically acceptable salt is anagrelide hydrochloride. In other embodiments, said phosphodiesterase 3A modulator compound is 3-hydroxy anagrelide or a pharmaceutically acceptable salt thereof. In further embodiments, said phosphodiesterase 3A modulator compound is DNMDP, BRD9500, BAY2666605, or a pharmaceutically acceptable salt thereof.

The present invention further relates to approaches for treating cancer in a subject. The approach can be used to treat any cancers or tumors, including both malignant and benign tumors, both primary tumors and metastases may be targets of the approach. In some embodiments, the treated cancer is selected from a group consisting of soft tissue sarcomas such as alveolar soft-part sarcoma, fibrosarcoma, myxofibrosarcoma, malignant fibro histiocytoma, gastrointestinal stromal tumor (GIST), liposarcoma, leiomyosarcoma, malignant peripheral nerve sheath tumor, rhabdomyosarcoma, undifferentiated and unclassified sarcomas, Ewing sarcoma, and synovial sarcoma. In some embodiments, the treated cancer is selected from a group consisting of schwannoma, glioblastoma, medulloblastoma, and meningioma. Preferably, the treated cancer is gastrointestinal stromal tumor (GIST) or liposarcoma.

In other embodiments, the treated cancer is selected from a group consisting of cancer of brain, oral cavity, the head and neck including the nasopharanygeal region, thyroid carcinoma, gastrointestinal cancers including oesophageal or gastric cancer, pancreatic, hepatocellular or colorectal cancer as well as cancer of the lungs and bronchus, and cancer of the ovaries, endometrium, cervix, breast, prostate, kidneys, skin mesothelioma, melanoma, Merkel cell carcinoma, gallbladder or multiple myeloma.

In other embodiments, the treated cancer can be pre-selected to be responsive to a PDE3A modulator. The pre-selection may be performed by detecting the expression of at least PDE3A and Schlafen 12 (SLFN12) polypeptide biomarkers relative to a reference. Preferably, said cancer selected as responsive to a PDE3A modulator is a bone, breast, cervical, colon, endometrium, GIST, head and neck, hematopoietic, kidney, liposarcoma, leiomyosarcoma, liver, lung, lymphoid, melanoma, ovarian, pancreas, prostate, soft-tissue sarcoma, thyroid cancer, or urinary tract cancer.

In further embodiments, the present disclosure is also directed to a method of treating cancer in a subject in need of such treatment comprising administering to the subject an effective amount of (a) a phosphodiesterase 3A modulator compound or a pharmaceutically acceptable salt thereof and (b) one or more inhibitors selected form the group consisting of: an inhibitor of Bcl-2 family proteins, a mTOR inhibitor, a histone deacetylase (HDAC) inhibitor, a DNA-dependent protein kinase (DNA-PK) inhibitor, an inhibitor of integrin alpha 2 protein, a NEDD8-activating enzyme (NAE) inhibitor, a PAK4 inhibitor and pharmaceutically acceptable salts thereof. In a preferred embodiment, a first dose of (a) and a first dose of (b) are simultaneously administered to the subject. In another preferred embodiment, the first dose of (a) and the first dose of (b) are administered sequentially in 24 hours, 2-5 days or a week.

The publications and other materials used herein to illuminate the background of the invention, and in particular, to provide additional details with respect to its practice, are incorporated herein by reference. The present invention is further described in the following Experimental section, which are not intended to limit the scope of the invention.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### EXPERIMENTAL SECTION

### Material and Methods

Unless otherwise stated, properties that have been experimentally measured or determined herein have been measured or determined at room temperature. Unless otherwise indicated, room temperature is 25° C. Unless otherwise stated, properties that have been experimentally measured or determined herein have been measured or determined at atmospheric pressure.

Cell lines. PDE3A-positive (GIST882, SA4, and GOT3) and PDE3A-negative cell lines (93TT449, 94T778, LPS141, MLS17656-92, MLS402-91, and SW872) were cultured in RPMI Medium 1640 (Gibco) with 5-, 10- or 20% fetal bovine serum (FBS, gibco), 100 U/mL of penicillin, 100 U/mL of streptomycin and 0.03 mg/mL of L-glutamine (Pen Strep Glut, Gibco, 10378-016) in a humidified, 5% CO2 atmosphere at 37° C.

High throughput drug sensitivity and resistance test. A sensitivity and resistance of cancer cell lines to 528 compounds were investigated in 9 cell lines (GIST882, SA4, GOT3, 93TT449, 94T778, LPS141, MLS17656-92, MLS402-91, and SW872). In addition, same compounds were investigated together with anagrelide hydrochloride (100 nM) treatment in the PDE3A low expressing cell lines SA4 and GOT3, and with BAY2666605 (40 nM) in all PDE3A-positive cell lines GIST882, SA4 and GOT3. The compounds were dissolved in 100% dimethyl sulfoxide or water and plated in 10-fold dilutions covering a 10,000-fold concentration range on flat clear bottom 384-well microplates as described in detail earlier (Pulkka O P, et al 2019). A cell viability was measured after 72 hours using CellTiter-Glo Cell Viability assay (Promega Inc.) and PHERAstar FS plate reader (BMG Labtech). The data were normalized to negative (0.01% dimethyl sulfoxide only) and positive (100 μmol/L benzethonium chloride) controls.

A four-parameter logistic dose-response curves were estimated by using the Marquardt-Levenberg algorithm and Breeze analysis platform. Drug responses to the test compounds were measured using the drug sensitivity score (DSS). Drug synergy partners, which were common in investigated cell lines were identified as following: DSS value of a single agent was subtracted from the DSS value of anagrelide-drug combination in single cell lines. Then average drug sensitivity score (aDSS) of the acquired DSS values (DSS combination—DSS anagrelide-drug combination) was calculated. aDSS values >3.0 were considered to indicate observed drug synergy in the cell lines. The analysis was repeated by using BAY2666605 as a PDE3A modulator.

Twelve drugs that showed synergy in the analyses were investigated more in detail in the GIST882, SA4, and GOT3 cell lines. Drug combination testing with anagrelide and 12 selected drugs was performed as described above but with selected drugs using drug concentration matrices covering seven different concentrations with 3 replicate sample of each combinations and in two replicate assays. Synergy was assessed using an R package SynergyFinder 2.0 (Ianevski A, et al. 2020). The tool implements synergy scoring with four major reference models: HSA, Loewe, Bliss and ZIP models. The degree of a drug combination effect can be readily visualized as a synergy landscape map over the dose matrix and ZIP synergy score.

Western blot. Western blotting was used to detect PDE3A and Schlafen 12 expression in cell lines by using a polyclonal rabbit anti-PDE3A antibody (dilution 1:1000; HPA014492, Sigma-Aldrich) and a polyclonal rabbit anti-SLFN12 (dilution 1:500, ab234418, Abcam), respectively. Cells were washed twice with cold PBS before lysed on ice in M-PER™ Mammalian Protein Extraction Reagent (Thermo Scientific™, 78501) containing HALT™ protease inhibitor cocktail (Thermo, 78429) and HALT™ phosphatase inhibitor cocktail (Thermo, 78420). Six to ten μg of protein lysate was separated using a gel electrophoresis and blotted onto PVDF membrane and then stained with antibodies. Immunostains were detected by a SuperSignal West Pico PLUS Chemiluminescent Substrate (Thermo Fisher Scientific Inc.) according to manufacturer's instructions.

Cell viability assay. Cells were plated on 96 Well White/Clear Tissue Culture Treated Plate (FALCON, 353377) 24 hours prior to medium change with drugs. Viabilities of cells in different treatments were evaluated 72 hours after treatment incubation with CellTiter-Glo® Luminescent Cell Viability Assay (G7572, Promega) following manufacturer's instruction. Reagent and cells stabilized to room temperature. Reagent added 100 μl/well, shaken for 2 minutes at room temperature and incubated for 10 min at room temperature before measuring the luminescence by using Hidex Sence microplate reader (Hidex oy). Growth medium was used to exclude background signal from the results.

Immunohistochemistry. Cell lines were grown up to 70% confluence, detached, counted (˜8 million cells/tube), washed twice with PBS (Corning, REF 21-040-CV) prior to splitting the cells into two Eppendorf tubes and centrifuged (10 000 rpm/˜9600 g) to form a dense cell pellet. The supernatant was removed, and the cell pellet was resuspended in 60 μl human plasma (+4° C.). Then, 60 ul of 100 NIH-U/ml thrombin (Merck, REF 1.12374.0001) at +4° C. was added to the cell suspension to clot the cells. Next, 1 ml of 5% formalin was added to detach the clots from the Eppendorf tubes, after which the clots were transferred to a larger tube containing ˜6 ml of 5% formalin. The cell clots were fixed in 5% formalin for 2 hours at room temperature. Following fixation, the cell clots were subjected to ascending alcohol/xylene series to dehydrate the samples. The cell clots were then immersed in paraffin overnight, forming FFPE-like histological samples.

Tissue slides were deparaffinized prior to immunohistochemical staining. An anti-PDE3A antibody (dilution 1:100; HPA014492, Sigma-Aldrich) was diluted in Draco antibody diluent (AD500, WellMed) incubated on slides 1 hour at a room temperature. Primary antibody binding was detected using a 1 Step Detection System, rabbit HRP (R500HRP, WellMed) and an ImmPact DAB Substrate kit (SK-4105, Vector). The slides were counterstained with Mayer's hematoxylin. The immunostaining of the cell line samples was graded as negative, low, intermediate or strong based on the intensity of staining.

### Results

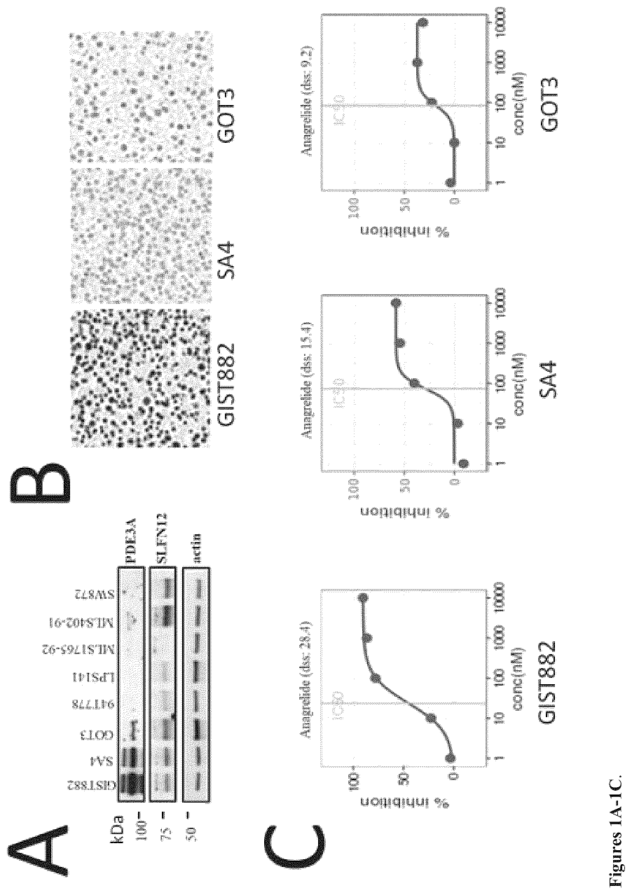

Expression of PDE3A, SLFN12 and actin was studied in eight cell lines to confirm the presence of these proteins (FIG. 1A). High-throughput drug screening of 528 compounds in these eight cell lines identified two cell lines, SA4 (IC50=73.5 nM; DSS=15.4) and GOT3 (IC50=84.3 nM; DSS=9.2), which were highly sensitive for Anagrelide treatment (FIG. 1C). Immunoblotting results verified that both cell lines express PDE3A and SLFN12 (FIG. 1B). However, PDE3A expression in the SA4 and GOT3 cell lines was significantly lower than in the GIST882 cell line. These three cell lines were used in the following experiments.

Next, we studied which drugs have synergy with anagrelide and BAY2666605 in the anagrelide sensitive cell lines by repeating the high-throughput screening for all compounds with anagrelide or BAY2666605. The synergy with anagrelide was found for 44 compounds in GOT3 cell line and SA4 cell line (Table 1). The synergy with BAY2666605 was found for 38 compounds in GIST882, GOT3 and SA4 cell lines (Table 2). Following synergistic compounds were discovered: Bcl-family inhibitors navitoclax, A-1155463, A-1331852, WEHI-539, venetoclax; PAK4 inhibitor PF-03758309; PI3K/AKT/mTOR pathway inhibitors serabelisib, GDC-0084, CC-223, CC-115 (also a DNA dependent protein kinase inhibitor), AZD8055, CUDC-907 (also a HDAC family inhibitor), temsirolimus, everolimus, sirolimus, ridaforolimus, uprosertib, dactolisib, copanlisib, omipalisib, LY3023414 (also a DNA dependent protein kinase inhibitor), NVP-BGT226; HDAC-family inhibitors quisinostat, pracinostat, AR-42, panobinostat, belinostat, givinostat, romidepsin, vorinostat, resminostat, abexinostat, rocilinostat, tubastatin A; NAE inhibitor pevonedistat; integrin alfa 2 inhibitor E7820; MELK inhibitor OTS163; p53-MDM2 pathway inhibitors SAR405853, AMG-232, idasanutlin; PIM kinase inhibitors PIM-447, ADZ1208; XPO1/CRM1 inhibitors eltanexor, Selinexor; aminopeptidase inhibitor tosedostat; Aurora B inhibitor AZD1152; IGFIR inhibitor BMS-754807; FAK inhibitor VS-4718; ATM inhibitor AZD0156; CDK2 inhibitor milciclib; CHEK1 inhibitor MK-8776; CENP-E inhibitor GSK923295; caseine kinase inhibitor PF-670426; proteosome inhibitor bortezomib; DOTL1 inhibitor pinometostat; and compounds midostaurin, temozolomide, bexarotene, ruboxitaurin and ONX-0914.

TABLE 1
Compounds having a synergistic effect with
Anagrelide in GOT3 and SA4 cell lines.

| | SA4 cell line: | GOT3 cell line: | |
|---|---|---|---|
| | DSS(combo | DSS(combo | Average |
| | (anagrelide))- | (anagrelide))- | DSS |
| Drug | DSS(drug) | DSS(drug) | (aDSS) |
| A-1155463 | 22.2 | 7.5 | 14.85 |
| E7820 | 18.2 | 7.5 | 12.85 |
| PF-03758309 | 20.1 | 3.7 | 11.9 |
| Navitoclax | 17.4 | 2.6 | 10 |
| A-1331852 | 14.9 | 3.6 | 9.25 |
| Quisinostat | 15.8 | 1.9 | 8.85 |
| Ridaforolimus | 13.2 | 4.4 | 8.8 |
| Pevonedistat | 13.8 | 2.7 | 8.25 |
| Pracinostat | 14.8 | −1.4 | 6.7 |
| Resminostat | 13.5 | −0.1 | 6.7 |
| Sirolimus | 9.5 | 3.4 | 6.45 |
| AR-42 | 12.9 | −0.8 | 6.05 |
| Panobinostat | 11.7 | −0.5 | 5.6 |
| Everolimus | 8.1 | 3 | 5.55 |
| Belinostat | 10 | 1.1 | 5.55 |
| OTS167 | 10.5 | 0.6 | 5.55 |
| Temsirolimus | 9.8 | 1.1 | 5.45 |
| CUDC-907 | 10.8 | 0 | 5.4 |
| PIM-447 | 7.9 | 2.5 | 5.2 |
| CC-115 | 9.5 | 0.8 | 5.15 |
| Abexinostat | 10.9 | −1.1 | 4.9 |
| Givinostat | 10 | −0.7 | 4.65 |
| AZD1152- | 5.9 | 3.4 | 4.65 |
| HQPA | | | |
| VS-4718 | 5.3 | 3.8 | 4.55 |
| WEHI-539 | 6.8 | 1.8 | 4.3 |
| SAR405838 | 7.8 | 0.1 | 3.95 |
| Serabelisib | 5.8 | 1.9 | 3.85 |
| Romidepsin | 6.3 | 1.4 | 3.85 |
| Tosedostat | 5.1 | 2.5 | 3.8 |
| Idasanutlin | 8.5 | −1 | 3.75 |
| Milciclib | 3.9 | 3.6 | 3.75 |
| AZD8055 | 5.9 | 1.5 | 3.7 |
| Midostaurin | 5.1 | 2.3 | 3.7 |
| AZD1208 | 7.1 | 0.3 | 3.7 |
| PF-670462 | 7.1 | 0.2 | 3.65 |
| Uprosertib | 5.4 | 1.8 | 3.6 |
| GSK923295 | 3.9 | 2.8 | 3.35 |
| Ruboxistaurin | 4.8 | 1.8 | 3.3 |
| Vorinostat | 7.2 | −0.7 | 3.25 |
| MK-8776 | 4.8 | 1.6 | 3.2 |
| CC-223 | 5.6 | 0.8 | 3.2 |
| Venetoclax | 6.2 | 0 | 3.1 |
| AMG-232 | 6.2 | 0 | 3.1 |
| GDC-0084 | 3.9 | 2.3 | 3.1 |

DSS = drug sensitivity scoring. aDSS = average of drug sensitivity scores. The combination was considered to show synergy, if aDSS value was higher than 3.0.

TABLE 2
Compounds having a synergistic effect with BAY2666605
in GIST882, GOT3 and SA4 cell lines.

| | GIST882 cell line: | SA4 cell line: | GOT3 cell line: | |
|---|---|---|---|---|
| | DSS(combo | DSS(combo | DSS(combo | Average |
| | (BAY2666605))- | (BAY2666605))- | (BAY2666605))- | DSS |
| Drug | DSS(drug) | DSS(drug) | DSS(drug) | (aDSS) |
| Navitoclax | 20.7 | 28.5 | 1.7 | 17.0 |
| Tosedostat | 21 | 14.6 | 6.7 | 14.1 |
| Venetoclax | 12.2 | 29 | 0.2 | 13.8 |
| Quisinostat | 8.7 | 16.2 | 8 | 11.0 |
| A-1331852 | 15.1 | 12.6 | 2.8 | 10.2 |
| Givinostat | 7.3 | 18.5 | 3 | 9.6 |
| A-1155463 | 23 | −3.6 | 6.9 | 8.8 |
| Bortezomib | 1.7 | 11.2 | 10.4 | 7.8 |
| Pevonedistat | 9.3 | 7 | 6.2 | 7.5 |
| Panobinostat | 6.1 | 6.7 | 8.2 | 7.0 |
| WEHI-539 | 16.1 | −1.1 | 5 | 6.7 |
| Everolimus | 3 | 12.4 | 4.1 | 6.5 |
| Vorinostat | 4.2 | 13.7 | 1.6 | 6.5 |
| Pracinostat | 5.7 | 12 | 1.5 | 6.4 |
| Idasanutlin | 0 | 19.6 | −0.5 | 6.4 |
| Abexinostat | 4.2 | 12.3 | 2.1 | 6.2 |
| Resminostat | 3.1 | 11.7 | 3.1 | 6.0 |
| Belinostat | −0.3 | 15.8 | 0.8 | 5.4 |
| Dactolisib | 3.1 | 4.6 | 8.2 | 5.3 |
| OTS167 | 1.9 | 10.5 | 3.2 | 5.2 |
| NVP-BGT226 | 6.2 | 8.1 | 0.1 | 4.8 |
| Temsirolimus | 7.4 | 5.3 | 1.2 | 4.6 |
| ONX-0914 | 8.4 | 4.6 | 0.9 | 4.6 |
| Pinometostat | 3.9 | 9.8 | 0 | 4.6 |
| Copanlisib | 6.8 | 8.1 | −1.4 | 4.5 |
| Omipalisib | 7.4 | 6.5 | −0.4 | 4.5 |
| Sirolimus | 3.4 | 7.9 | 2 | 4.4 |
| Tubastatin A | −0.3 | 10.7 | 2.8 | 4.4 |
| LY3023414 | 2.4 | 9.3 | 1.5 | 4.4 |
| BMS-754807 | 0.7 | 7.5 | 4.4 | 4.2 |
| Selinexor | 7.9 | 5.4 | −0.7 | 4.2 |
| Temozolomide | 5.4 | 5.2 | 1.9 | 4.2 |
| Ridaforolimus | 0.3 | 13.7 | −1.9 | 4.0 |
| Bexarotene | 6.3 | 6.4 | −1.1 | 3.9 |
| PF-03758309 | 0.5 | 13.4 | −3.6 | 3.4 |
| Rocilinostat | 1.4 | 7.5 | 0.9 | 3.3 |
| Eltanexor | 5.4 | 3.1 | 0.8 | 3.1 |
| AZD0156 | 7.5 | 3 | −1.2 | 3.1 |

DSS = drug sensitivity scoring. aDSS = average of drug sensitivity scores. The combination was considered to show synergy, if aDSS value was higher than 3.0.

The twelve compounds which showed synergy with anagrelide and/or BAY2666605 in the analyses were investigated further in GOT3, SA4 and GIST882 cell lines by using drug concentration matrices covering seven different concentrations of anagrelide and the agent of interest. The synergy results were verified for all investigated drugs in all three cell lines (Table 3).

TABLE 3
Average ZIP synergy scores in GIST882,
SA4 and GOT3 cell lines.

| Compound | GIST882 | SA4 | GOT3 |
|---|---|---|---|
| A1155463 | 15.5 | 11.1 | 12.7 |
| A1331852 | 9.6 | 11.1 | 11.6 |
| Navitoclax | 11.7 | 8 | 2.4 |
| Ridaforolimus | 3.9 | 6.2 | 1.8 |
| Everolimus | 5 | 5.5 | 2.3 |
| Sirolimus | 1.1 | 7.9 | 0.4 |
| Temsirolimus | 2.8 | 3.6 | 0.4 |
| CC-115 | 3.1 | 4.1 | 1.6 |
| PF-03758309 | 11.5 | 16.8 | 8.5 |
| E7820 | 3 | 14.8 | 1.6 |
| Quisinostat | 6.5 | 8.7 | 6.9 |
| Pevonedistat | 4.6 | 7.8 | 12.7 |

ZIP = zero interaction potency.

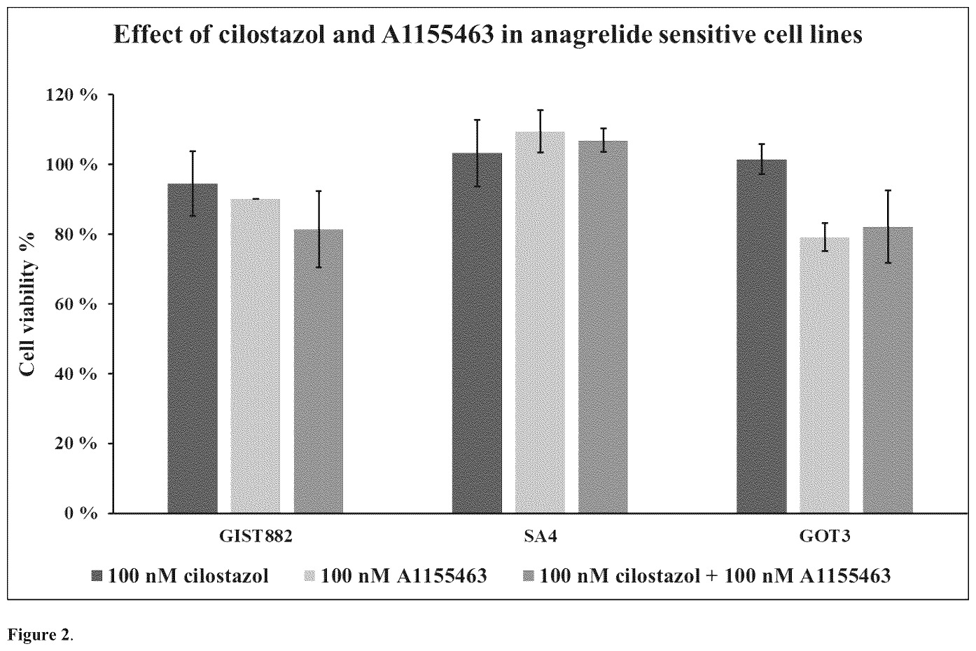

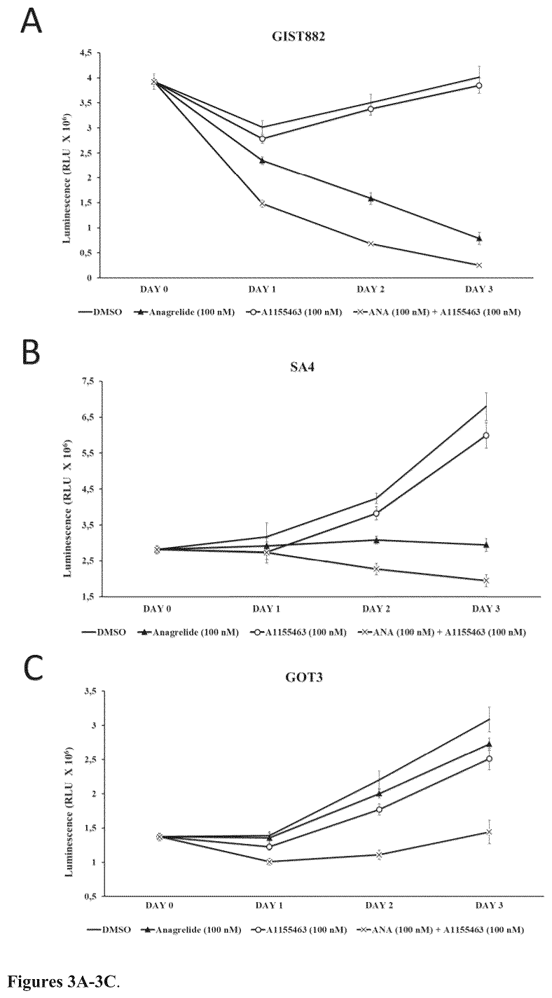

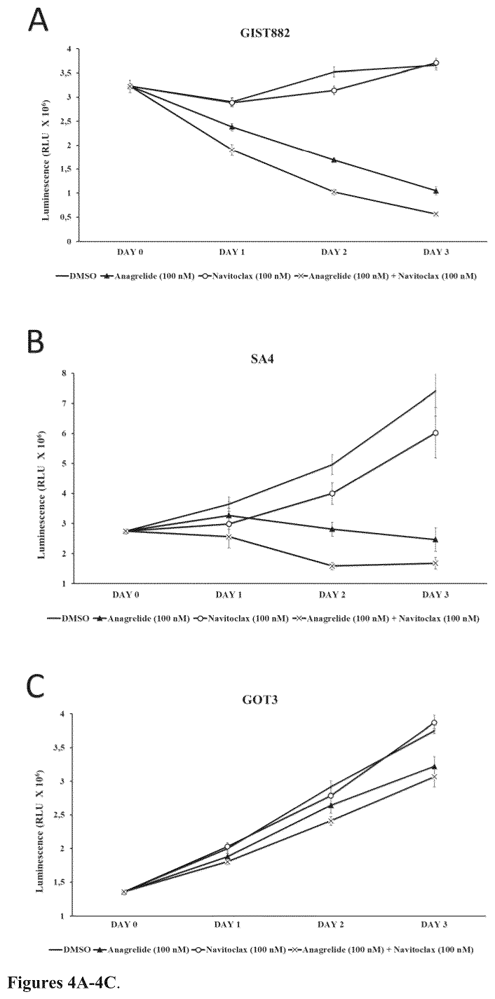

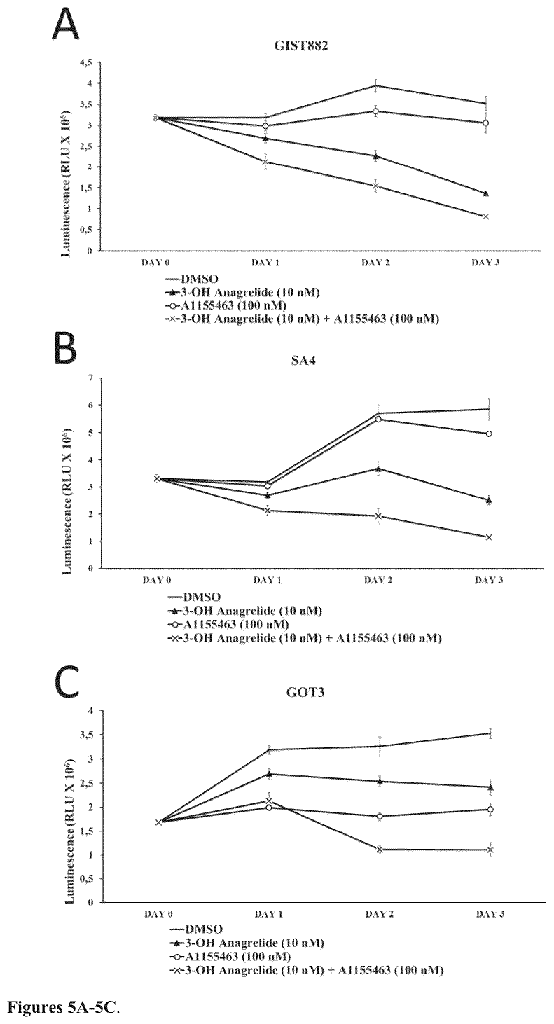

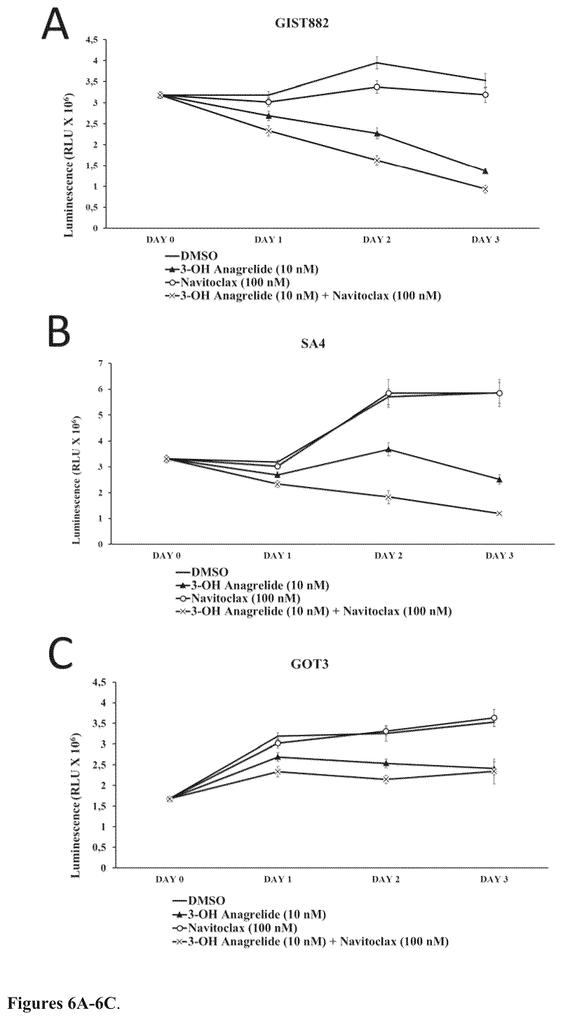

Bcl-family inhibitors navitoclax, A-1155463 and A-1331852 showed significant efficacy in cell lines only when they were administered with Anagrelide when they were analyzed by drug concentration matrices and SynergyFinder 2.0. A cell viability assay verified the result when cells were treated with navitoclax or A-1155463 and with Anagrelide (FIGS. 3 and 4) or with 3-hydroxy-anagrelide (FIGS. 5 and 6). The observed synergy between PDE3A modulator anagrelide and A-1155463 was not however achieved with a PDE3A inhibitor cilostazol, indicating that formation of PDE3A-SLFN12 interaction is required to induce PDE3A-related anticancer effect on cells (FIG. 2).

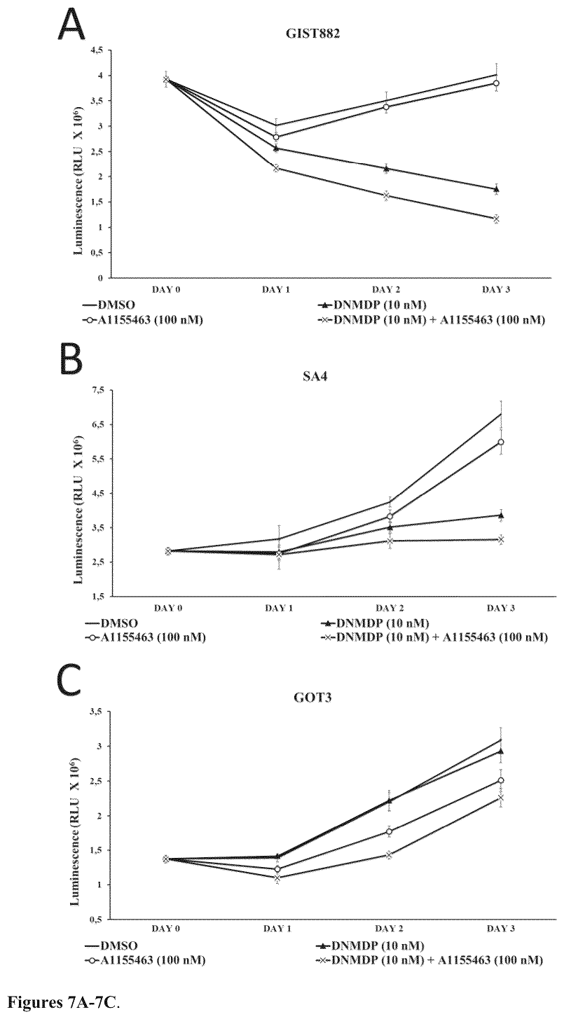

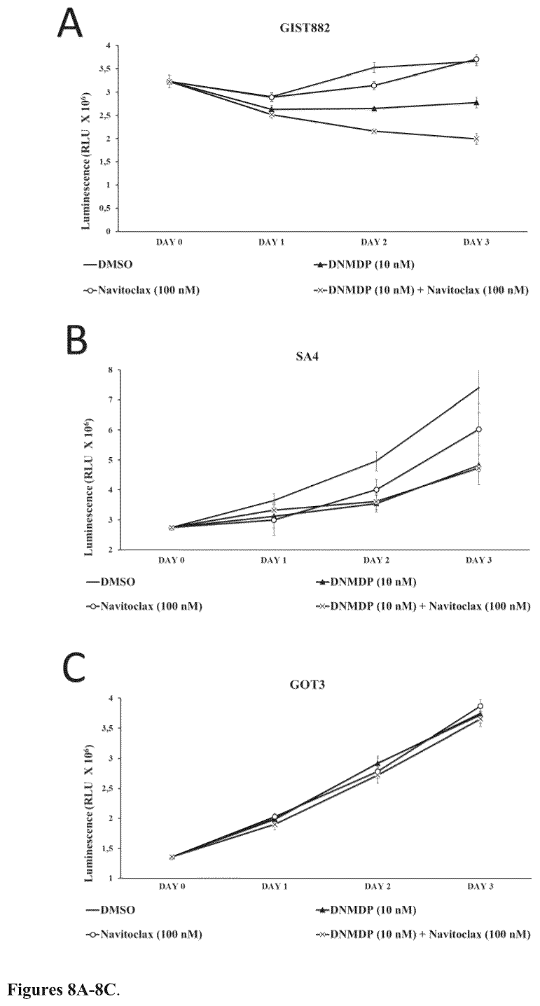

Finally, we investigated, whether the observed synergy is specific to anagrelide or whether other PDE3A modulators have the same effect. A1155463 and navitoclax both showed synergy with DNMDP in cell lines indicating that the discovered synergy is not specific to anagrelide (FIGS. 7-8). Based on these results, other PDE3A-specific agents, which induce formation of PDE3A-Schlafen 12 interaction, can be used in cancer combination therapy.

### CONCLUSION

We have discovered synergy between PDE3A-specific agents and Bcl-family, PI3K/AKT/mTOR, a DNA-dependent protein kinase, Integrin alpha 2, HDAC-family, NEDD8-activating enzyme and PAK4 inhibitors. Combination therapy of PDE3A-specific agents and inhibitors mentioned above offers novel therapies to treat PDE3A-positive cancers.

### CITATION LIST

### Patent Literature

EP3411037
WO2015055898

### Non Patent Literature

An R. Liu J, He J, Wang F, Zhang Q, Yu Q. PDE3A inhibitor anagrelide activates death signaling pathway genes and synergizes with cell death-inducing cytokines to selectively inhibit cancer cell growth. Am J Cancer Res 2019, 9; 1905-1921.
de Waal L, Lewis T A, Rees M G, Tsherniak A, Wu X, Choi P S, Gechijian L, Hartigan C, Faloon P W, Hickey M J, Tolliday N, Carr S A, Clemons P A, Munoz B, Wagner B K, Shamji A F, Koehler A N, Schenone M, Burgin A B, Schreiber S L, Greulich H, Meyerson M. Identification of cancer-cytotoxic modulators of PDE3A by predictive chemogenomics. Nat Chem Biol 2016, 12; 102-8.
Garvie C W, Wu X, Papanastasiou M, Lee S, Fuller J, Schnitzler G R, Horner S W, Baker A, Zhang T, Mullahoo J P, Westlake L, Hoyt S H, Toetzl M, Ranaghan M J, de Waal L, McGaunn J, Kaplan B, Piccioni F, Yang X, Lange M, Tersteegen A, Raymond D, Lewis T A, Carr S A, Cherniack A D, Lemke C T, Meyerson M, Greulich H. Structure of PDE3A-SLFN12 complex reveals requirements for activation of SLFN12 RNase. Nat Commun 2021, 12, 4375.
Ianevski A, Giri A K, Aittokallio T. SynergyFinder 2.0: visual analytics of multi-drug combination synergies. Nucleic Acids Res 2020, 48 (W1); W488-W493.
Lewis T A, de Waal L, Wu X, Youngsaye W, Wengner A, Kopitz C, Lange M, Gradl S, Ellermann M, Lienau P, Schreiber S L, Greulich H, and Meyerson M, Optimization of PDE3A Modulators for SLFN12-Dependent Cancer Cell Killing, ACS Med. Chem. Lett. 2019, 10, 1537-1542.
Nazir M, Senkowski W, Nyberg F, Blom K, Edqvist P H, Jarvius M, Andersson C, Gustafsson M G, Nygren P, Larsson R, Fryknäs M. Targeting tumor cells based on Phosphodiesterase 3A expression. Exp Cell Res 2017, 36; 308-315.
Pulkka O P, Gebreyohannes Y K, Wozniak A, Mpindi J P, Tynninen O, Icay K, Cervera A, Keskitalo S, Murumagi A, Kulesskiy E, Laaksonen M, Wennerberg K, Varjosalo M, Laakkonen P, Lehtonen R, Hautaniemi S, Kallioniemi O, Schöffski P, Sihto H, Joensuu H Anagrelide for Gastrointestinal Stromal Tumor. Clin Cancer Res 2019, 25:1676-1687.
Wu X, Schnitzler G R, Gao G F, Diamond B, Baker A R, Kaplan B, Williamson K, Westlake L, Lorrey S, Lewis T A, Garvie C W, Lange M, Hayat S, Seidel H, Doench J, Cherniack A D, Kopitz C, Meyerson M, Greulich H. Mechanistic insights into cancer cell killing through interaction of phosphodiesterase 3A and schlafen family member 12. J Biol Chem 2020, 295; 3431-3446.

1-30. (canceled)

31. A method of treating cancer in a subject in need of such treatment comprising:
administering to the subject an effective amount of (a) a phosphodiesterase 3A modulator compound or a pharmaceutically acceptable salt thereof; and (b) one or more inhibitors selected from the group consisting of: an inhibitor of Bcl-2 family proteins, a mTOR inhibitor, a histone deacetylase (HDAC) inhibitor, a DNA-dependent protein kinase (DNA-PK) inhibitor, an inhibitor of integrin alpha 2 protein, a NEDD8-activating enzyme (NAE) inhibitor, a PAK4 inhibitor, and pharmaceutically acceptable salts thereof.

32. The method according to claim 31, wherein said phosphodiesterase 3A modulator compound is selected from the group consisting of: anagrelide, 3-hydroxy anagrelide, nauclefine, BRD9500, DNMDP, (R)-DNMDP, BAY2666605, and pharmaceutically acceptable salts thereof.

33. The method according to claim 31, wherein said inhibitor of Bcl-2 family proteins is selected from the group consisting of: A-1155463, ABT-263 (navitoclax), A-1331852, AT-101, WEHI-539, gambogic acid, A-1210477, (S)-gossypol acetic acid, apogossypol, ABT-737, and pharmaceutically acceptable salts thereof.

34. The method according to claim 31, wherein said phosphodiesterase 3A modulator compound is anagrelide, 3-hydroxy anagrelide, BRD9500, BAY2666605, or a pharmaceutically acceptable salt thereof and the inhibitor of Bcl-2 family proteins is A-1155463, ABT-263 (navitoclax), or a pharmaceutically acceptable salt thereof.

35. The method according to claim 31, wherein the mTOR inhibitor is selected from the group consisting of rapamycin, sirolimus, everolimus, ridaforolimus, temsirolimus, CC-115, Torin 1, Torin 2, and pharmaceutically acceptable salts thereof.

36. The method according to claim 31, wherein said phosphodiesterase 3A modulator compound is anagrelide, 3-hydroxy anagrelide, BRD9500, BAY2666605, or a pharmaceutically acceptable salt thereof and the mTOR inhibitor is ridaforolimus, temsirolimus, sirolimus, everolimus, or a pharmaceutically acceptable salt thereof.

37. The method according to claim 31, wherein the histone deacetylase (HDAC) inhibitor is selected from the group consisting of: vorinostat, entinostat, panobinostat, mocetinostat, belinostat, ricolinostat, romidepsin, givinostat, dacinostat, quisinostat, pracinostat, resminostat, droxinostat, abexinostat, RGFP966, AR-42, PCI34051, trichostatin A, SB939, CI994, CUDC-907, tubacin, chidamide, RG2833, M344, MC1568, tubastatin A, scriptaid, valproic acid, sodium phenylbutyrate, tasquinimod, kevetrin, HPOB, 4SC-202, TMP269, CAY10603, BRD73954, BG45, LMK-235, nexturastat A, CG200745, CHR2845, CHR3996, and pharmaceutically acceptable salts thereof.

38. The method according to claim 31, wherein said phosphodiesterase 3A modulator compound is anagrelide, 3-hydroxy anagrelide, BRD9500, BAY2666605, or a pharmaceutically acceptable salt thereof and the HDAC inhibitor is quisinostat, or a pharmaceutically acceptable salt thereof.

39. The method according to claim 31, wherein the DNA-dependent protein kinase (DNA-PK) inhibitor is selected from the group consisting of: NU7441, NU7026, KU-0060648, LTURM34, CC-115, PIK-90, Wortmannin, LY3023414, M3814, SF2523, compound 401, and pharmaceutically acceptable salts thereof.

40. The method according to claim 31, wherein said phosphodiesterase 3A modulator compound is anagrelide, 3-hydroxy anagrelide, BAY2666605, or a pharmaceutically acceptable salt thereof and the DNA-PK inhibitor is CC-115, or a pharmaceutically acceptable salt thereof.

41. The method according to claim 31, wherein said phosphodiesterase 3A modulator compound is anagrelide, 3-hydroxy anagrelide, BRD9500, BAY2666605, or a pharmaceutically acceptable salt thereof and the inhibitor of integrin alpha 2 protein is E7820 or a pharmaceutically acceptable salt thereof.

42. The method according to claim 31, wherein said phosphodiesterase 3A modulator compound is anagrelide, 3-hydroxy anagrelide, BRD9500, BAY2666605, or a pharmaceutically acceptable salt thereof and the NAE inhibitor is pevonedistat (MLN4924) or a pharmaceutically acceptable salt thereof.

43. The method according to claim 31, wherein the PAK4 inhibitor is selected from the group consisting of: KPT-9274, PF-03758309, IPA-3, FRAXIQ36, LCH-7749944, glaucambinone, KY-04031, KY-04045, Inkal, GL-1196, GNE-2861, and pharmaceutically acceptable salts thereof.

44. The method according to claim 31, wherein said phosphodiesterase 3A modulator compound is anagrelide, 3-hydroxy anagrelide, BRD9500, BAY2666605, or a pharmaceutically acceptable salt thereof and the PAK4 inhibitor is PF-03758309 or a pharmaceutically acceptable salt thereof.

45. The method according to claim 31, wherein i) a first dose of the phosphodiesterase 3A modulator compound and a first dose of the one or more inhibitors are simultaneously administered to the subject, or ii) wherein a first dose of the phosphodiesterase 3A modulator compound and a first dose of the one or more inhibitors are administered sequentially.

46. The method according to claim 31, wherein the phosphodiesterase 3A modulator compound and the one or more inhibitors are formulated in one pharmaceutical composition or separate pharmaceutical compositions, and wherein said composition(s) comprise(s) at least a pharmaceutically acceptable buffer, carrier, preservative, or adjuvant.

47. The method according to claim 31, wherein said pharmaceutical composition(s) is/are formulated for sustained-release, delayed-release, or timed-release, or said pharmaceutical composition(s) is/are a blend of sustained-release and immediate-release formulations.

48. The method according to claim 31, wherein the treated cancer is a soft-tissue sarcoma selected from the group consisting of: alveolar soft-part sarcoma, fibrosarcoma, gastrointestinal stromal tumor (GIST), liposarcoma, leiomyosarcoma, malignant fibro histiocytoma, malignant peripheral nerve sheath tumor, rhabdomyosaroma, myxofibrosarcoma, undifferentiated and unclassified sarcomas, Ewing sarcoma, and synovial sarcoma.

49. The method according to claim 31, wherein the treated cancer is a cancer pre-selected as a cancer that is responsive to a PDE3A modulator, and wherein said pre-selection is performed by detecting the expression of at least PDE3A and Schlafen 12 (SLFN12) polypeptide biomarkers relative to a reference.

50. The method according to claim 49, wherein said cancer selected as responsive to a PDE3A modulator is a bone, breast, cervical, colon, endometrium, GIST, head and neck, hematopoietic, kidney, liposarcoma, leiomyosarcoma, liver, lung, lymphoid, melanoma, ovarian, pancreas, prostate, soft-tissue sarcoma, thyroid cancer, or urinary tract cancer.
